(19) United States
(12) Patent Application Publication
(10) Pub. No.: US 2026/0151276 A1
(43) Pub. Date: Jun. 4, 2026

(54) Diaper Cores

(71) Applicant: Freestyle World, Inc., Santa Monica, CA (US)

(72) Inventors: Russ Wallace, Santa Clara, CA (US); Michael Constantiner, Newport Beach, CA (US)

(73) Assignee: Freestyle World, Inc., Santa Monica, CA (US)

(21) Appl. No.: 19/289,012

(22) Filed: Aug. 2, 2025

Related U.S. Application Data
(60) Provisional application No. 63/727,107, filed on Dec. 2, 2024.

(51) Int. Cl. A61F 13/53; A61F 13/513

(52) U.S. Cl. CPC A61F 13/53; A61F 13/513; A61F 2013/51355; A61F 2013/530693

(57) ABSTRACT

Described herein are examples of highly absorbent diaper cores including a top sheet, a super absorbent matrix, and a pulp layer. The super absorbent matrix can include a first non-woven distribution layer, a first super absorbent polymer layer, a cellulosic layer, a second super absorbent polymer layer, and a second non-woven distribution layer.

### CROSS REFERENCE TO RELATED APPLICATIONS

The present application claim priority to U.S. Provisional Patent Application No. 63/727,107 entitled “DIAPER CORES” filed on Dec. 2, 2024. The entire contents of the above-listed application are hereby incorporated by reference for all purposes.

### BACKGROUND

The present invention relates to the field of disposable wearable articles, and particularly the core absorption components of these articles. Highly absorbent articles are designed to assist in avoiding leakage of bodily fluids and semi-fluids, such as urine, menses and related compositions.

### BRIEF DESCRIPTION OF THE DRAWINGS

The present description will be understood more fully when viewed with the accompanying drawings of various examples of diaper cores. The description is not meant to limit the diaper cores to specific examples. Rather, the specific examples depicted and described are provided for explanation and understanding of diaper cores. The drawings may be referred to as drawings, figures, and/or FIGs throughout the description.

FIG. 1 illustrates a diaper core, according to an embodiment.

### DETAILED DESCRIPTION

Diaper cores as disclosed herein will become better understood through a review of the following detailed description in conjunction with the figures. The detailed description and figures provide merely examples of the various embodiments of the diaper cores. Many variations are contemplated for different applications and design considerations; however, for brevity and clarity, all the contemplated variations may not be individually described in the following detailed description. Those skilled in the art will understand how the disclosed examples may be varied, modified, and altered and not depart in substance from the scope of the examples described herein.

A conventional diaper core may include a top sheet, pulp and polymer components. These conventional diaper cores have been used for decades and have the known draw backs of placing pulp close to or directly against the skin of the wearer. Further, these traditional diaper cores can have leakage resulting in the wearer (often an infant) being wet and uncomfortable. This can cause rashes, itching and general discomfort for the wearer.

Existing diaper cores can include a mixture of pulp and polymers distributed randomly throughout the core. Alternatively, these diapers can also include an acquisition layer, a distribution layer and/or a top sheet to assist with water retention and/or skin protection. Known top sheets are generally prepared from nonwoven materials. While these diapers can provide limited water retention and skin protection, more efficient products are needed.

Implementations of highly absorbent and retentive diaper cores may address some or all of the problems described above, and such implementations may enhance the comfort of the wearer, and the length of the time the disposable wearable articles, such as a diaper for an infant, can be worn.

Embodiments of highly absorbent and retentive diaper cores may include a multilayer system that includes a top sheet, a multi-layered matrix (or super absorbent polymer matrix “SAP matrix”), and a pulp layer. The multilayer matrix can include three to ten layers, four to six layers, and/or five layers. The layers can include a first nonwoven distribution layer, a first super absorbent polymer layer, a cotton layer, a second super absorbent polymer layer, and a second nonwoven distribution layer.

The configuration of the multilayer system according to the present invention provides improved water retention in the diaper. Further, the multilayer system induces greater water retention in the pulp layer, added retention in the super absorbent polymer, and lower rates of irritation for the wearer.

Embodiments of the pulp layer of the present invention can use about 50% less pulp than conventional diaper cores due to the exceptional absorption of the SAP matrix. The reduction in size and weight of the pulp layer provides a smaller, thinner and lighter diaper. This can provide added benefits in appearance for the wearer, and reduced waste in disposal of the diaper. Further, the reduced size of the pulp layer and associated pulp material reduces the cost of the diaper as well as shipping and packaging size.

The SAP matrix of the present invention can create a one-way permeable moisture barrier at the top of the diaper core, directly underneath the top sheet. The SAP matrix allows moisture into the diaper core, but because the moisture immediately activates the SAP in the SAP matrix on contact, the SAP blocks the moisture from leaking out of the core, onto the top sheet, and into contact with the user's skin.

The present invention further solves one of the known problems with super absorbent polymers, namely that they can act (absorb) water slowly. The present invention solves this problem with a diaper core configuration in which water (or other liquid or semi-liquid materials) pass into the SAP matrix, and while the water is being absorbed by the SAP materials, excess water passes into the pulp layer. The pulp layer then holds any excess water, while the SAP matrix forms a shield or barrier above the pulp layer, stopping any water from re-exiting (rewetting) the diaper core (potentially onto the skin of the user). This allows the diaper cores of the present invention to cause any moisture that enters the diaper to be locked in the diaper core, away from the skin of the user.

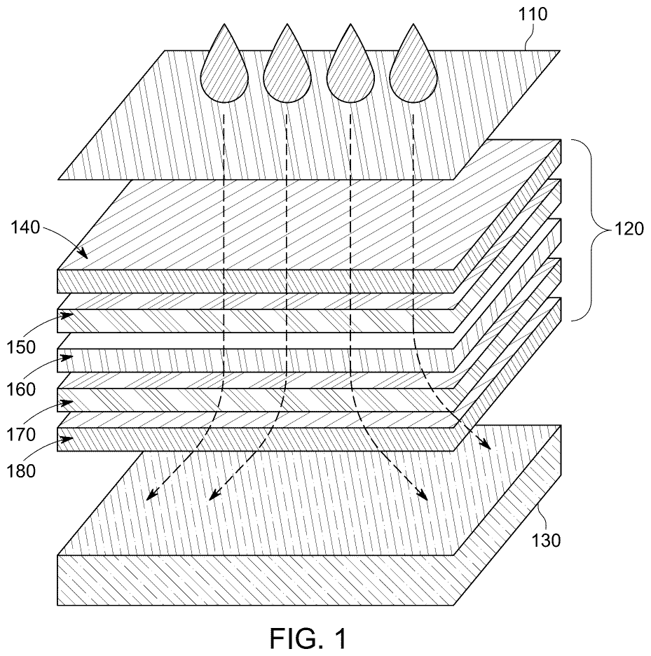

FIG. 1 illustrates a diaper core, according to an embodiment. The diaper core 100 can include a top sheet 110, a super absorbent polymer (SAP) matrix 120, and a pulp layer 130.

The SAP matrix 120 can further include a first non-woven distribution layer 140, a first super absorbent layer 150, a cellulosic layer 160 made from cotton, a second super absorbent layer 170, and a second non-woven distribution layer 180.

The top sheet 110 of the present invention can generally be prepared from nonwoven materials and can be prepared from hydrophilic and permeable materials. The hydrophilic nature of the top sheet helps to reduce wetness to the user and avoid liquid run off out the absorbent article. The top sheet allows the liquid waste to pass through the top sheet into SAP polymer matrix 120, and then into the pulp layer 130. Embodiments of the top sheet 110 can be provided with holes or apertures to assist with the transport of the liquid to the super absorbent matrix 120.

Examples of materials suitable for the top sheet include, but are not limited to, nonwoven fabrics often formed by processes such as meltblowing, spunbonding, solvent spinning, electrospinning, and carding. The basis weight of nonwoven fabrics is usually expressed in grams per square meter (g/m²). Top sheet material can be prepared from a combination of polyethene, polypropylene and polyethylene terephthalate. Embodiments of the top sheet material can have a density of 10 to 100 g/m², a density of 25 to 70 g/m², and a density of at least 38 g/m².

The SAP matrix 120 can be a portion of diaper core that absorbs and retains the liquid and semi-liquid bodily fluids. The SAP matrix can be prepared from multiple layers of materials. Embodiments can include a five-layer structure that includes a first non-woven distribution layer 140, a first SAP layer 150, a cellulosic layer or cotton layer 160, a second SAP layer 170, and a second non-woven distribution layer 180.

The top sheet can have a thickness between 0.07 mm and 0.13 mm, between 0.8 mm and 0.12 mm, and between 0.09 mm and 0.11 mm. The top sheet can be between 0.6% and 1.4% weight percent of the diaper core, between 0.07% and 1.3% weight percent of the diaper core, and between 0.08% and 1.2% weight percent of the diaper core.

The SAP matrix can have a total thickness between about 2.5 mm and 4 mm, between about 2.8 mm and 3.2 mm, and between about 2.9 mm and 3 mm. The SAP matrix can be between about 10 and 45 weight percent of the diaper core, between about 20 and 40 weight percent of the diaper core, and between about 25 and 35 weight percent of the diaper core.

Below the SAP matrix 120 can be a pulp layer 130. The pulp layer 130 can be an additional layer of protection and absorption capacity for the diaper core. The present invention allows the diaper core to include up to and including 50% or less pulp material than conventional diaper cores. This results in less material required for the production of the diaper, less waste when disposing the diaper, and a slimmer and more comfortable fit for the wearer. The pulp material can be chlorine free.

The pulp material in the pulp layer 130 and/or the cellulosic layer 160 can be prepared from various cellulosic materials. Examples include comminuted wood pulp, often referred to as “air felt” or “fluff,” tissue, tissue wraps, tissue laminates, cotton, creped cellulose wadding, melt blown polymers, chemically stiffened cellulosic fibers, modified cellulosic fibers, cross-linked cellulosic fibers, absorbent foams, absorbent sponges, super absorbent polymers, superabsorbent fibers, absorbent gelling materials, and combinations thereof.

The pulp layer 130 can have a thickness between about 4 mm to about 10 mm, between about 5 mm to about 9 mm, and between about 6 mm to about 8 mm. Embodiments of the pulp layer can be between about 50 to about 90 weight percentage of the diaper core, between about 60 to about 80 weight percentage of the diaper core, and between about 65 to about 70 weight percentage of the diaper core.

The super absorbent polymers (SAP) of the present invention can include polymeric materials that are water-insoluble and water swellable. These polymers are capable of absorbing high volumes of fluid in relation to their weight and can retain liquid under pressure. Examples of super absorbent polymers include, but are not limited to, acrylic acid copolymers, saponified acrylic acid ester-vinyl acetate copolymer, hydrolyzed starch-acrylonitrile graft copolymer, acrylamide copolymer, neutralized starch-acrylic acid graft copolymer, hydrolyzed acrylonitrile copolymer, modified cross-linked polyvinyl alcohol, neutralized self-cross-linking polyacrylic acid, cross-linked polyacrylate salt, carboxylated cellulose, cross-linked polyacrylates including sodium polyacrylate and polyacrylamides; graft copolymers on polysaccharides such as chitin, chitosan, cellulose, starch, natural gums and polypeptide-based copolymers such as saponified starch-graft polyacrylonitrile, hydroxypropyl-cellulose (HPC); hydroxypropylmethylcellulose (HPMC), carboxymethyl-cellulose (CMC); carboxymethyl starch (CMS); cross-linked copolymers of maleic anhydride; polyvinyl alcohol; polyvinyl cthers; polymers and copolymers of vinyl sulfonic acid; copolymer, and neutralized crosslinked isobutylene-maleic anhydride copolymer.

The super absorbent polymer can be prepared in various forms, including particles, beads, agglomerations, granules, spheres, flakes, fibers, and amorphous particles. Embodiments can include the super absorbent polymer in agglomerations or granules which can be prepared in layers.

The super absorbent polymers according to the present invention can be distributed in separate layers, such as a first SAP layer and a second SAP layer in the SAP matrix of the diaper core. The total weight of both (two) SAP layers can be present at about 1 to about 20 weight percentage of the SAP matrix, at about 3 to about 15 weight percentage of the SAP matrix, and at about 5 to about 10 weight percentage of the SAP matrix. Individual SAP layers, i.e., the first and second SAP layers can each be present at about 0.5 to about 10 weight percentage of the SAP matrix, at about 1.5 to about 7.5 weight percentage of the SAP matrix, and at about 2.5 to about 5 weight percentage of the SAP matrix. The super absorbent polymer can be a combination of two commercial grade superabsorbent polymers wherein both are acrylic acid copolymers, or polymers of acrylic acid, such as those available from BASF (e.g., Hysorb N7059) and Sumitomo (e.g., Aqua Keep SA605).

Embodiments of the SAP layers 150/170 can be prepared by forming a layer of polymeric material, e.g., powder, on top of and/or below the cellulosic material. This can form a fibrous web holding the individual SAP particles in place forming an SAP layer 150/170.

The SAP layers 150/170 individually can have a thickness between about 0.02 mm to about 0.1 mm, between about 0.04 mm to about 0.08 mm, between about 0.02 mm to about 0.8 mm, and between about 0.05 mm to about 0.06 mm.

Cellulosic materials used in the cellulosic layer 160 (or pulp layer 130) as used in the present invention can refer to cellulosic fluff pulps that are capable of forming a web structure to aid in absorption. Examples of cellulosic fluff pulps are softwood pulp including, but not limited to, southern pine, western hemlock, white pine, Caribbean pine, spruce, Douglas fir; hard wood pulp including, but not limited to, gum, maple, oak, aspen eucalyptus, poplar, beech; cellulosic fiber including, but not limited to cotton linter, flax, bamboo, bagasse, grass and kemp; and combinations thereof. Embodiments can include total chlorine free cellulosic fluff pulp.

The cotton or cellulosic layer 160 according to the present invention can be present at about 60-95% by weight of the SAP matrix, at about 65-90% by weight of the SAP matrix, and at about 70-85% by weight of the SAP matrix.

The cotton or cellulosic layer 160 can have a thickness between about 1 mm to about 3.5 mm, between about 1.5 mm to about 3 mm, and between about 2 mm to about 2.5 mm.

The cellulosic materials can be pretreated prior to their use in the diaper core. Examples of pretreatment include physical treatment such as steam, and chemical pretreatment, such as thermomechanical pulp treatment. The cellulosic fibers can be obtained and processed for use in the present invention in the form of cellulose fibers in sheet, roll, or fluff form. Further, the cellulosic material can be provided as a mat of nonwoven material, for example, as a thermal bonded non-woven mat, or a stabilized resin-bonded material. In a common application, a sheet of cellulosic material can be pulverized in a hammermill or fiberizer to create distinct and shorter fibers. These short fibers can be combined with superabsorbent polymers through gravity, vacuum, or positive air pressure to form the absorbent core.

The first and second non-woven distribution layers 140/180 can assist with the fluid management in the absorbent article. The distribution layers can be prepared from a hydrophilic material. The distribution layers can assist in the wicking of liquids and spreading of moisture across the SAP matrix and the pulp layer of the diaper core so as to maximize the utilization of all SAP in the core. The distribution layer can include an open structure with high bulk to provides for rapid liquid distribution and absorption. Examples of materials suitable for the distribution layer can include, but are not limited to, cellulosic materials cross-linked with dimethyl dihydroxyethylene urea or alkane polycarboxylic acids, and polyethylene/polypropylene bicomponent fibers.

Embodiments of the first and second non-woven distribution layers 140/180 can include a combination of polyethylene and polypropylene, also known as a bicomponent fiber. The distribution layer can have a density of 10 to 90 g/m², preferably a density of 20 to 60 g/m², and most preferably a density of at least 35 g/m².

The first and second non-woven distribution layers 140/180 can individually have a thickness between about 0.1 mm to about 0.7 mm, between about 0.3 mm and 0.5 mm, and between about 0.4 mm to about 0.45 mm. Embodiments of the first and second non-woven distribution layers can individually be between about 5 to about 20 weight percentage of the SAP Matrix, between about 7 to about 15 weight percentage of the SAP Matrix, and between about 10 to about 12 weight percentage of the SAP Matrix.

Embodiments of the pulp layer 130 can include wood pulp and mixtures of wood pulp and other cellulosic materials as the absorbent material in the pulp layer. Further the pulp layer can optionally contain an odor reducing agents, such as activated charcoal admixed in, or distributed with, the wood pulp in the pulp layer.

Table I provides ranges for the width and weight as a percentage of the diaper core for the top sheet, SAP Matrix and Pulp Layer.

TABLE I

| | First | Second | Third | First | Second | Third |
|---|---|---|---|---|---|---|
| Component of | Range | Range | Range | Range | Range | Range |
| Diaper Core | (mm) | (mm) | (mm) | (wt %) | (wt %) | (wt %) |
| Top Sheet | 0.07-0.13 | 0.8-0.12 | 0.09-0.11 | 0.6-1.4 | 0.07-1.3 | 0.08-1.2 |
| SAP Matrix | 2.5-4 | 2.8-3.2 | 2.9-3 | 10-45 | 20-40 | 25-35 |
| Pulp Layer | 4-10 | 5-9 | 6-8 | 50-90 | 60-80 | 65-70 |

Table II below provides ranges for the width and weight as a percentage of the SAP matrix for the first and second non-woven distribution layers, the first and second super absorbent layers, and the cellulosic layer.

TABLE II

| | First | Second | Third | First | Second | Third |
|---|---|---|---|---|---|---|
| Component of | Range | Range | Range | Range | Range | Range |
| SAP Matrix | (mm) | (mm) | (mm) | (wt %) | (wt %) | (wt %) |
| First non-woven | 0.1-0.7 | 0.3-0.5 | 0.4-0.45 | 5-20 | 7-15 | 10-12 |
| distribution layer | | | | | | |
| First super | 0.2-0.1 | 0.04-0.08 | 0.05-0.06 | 0.5-10 | 1.5-7 | 2.5-5 |
| absorbent layer | | | | | | |
| Cellulosic layer | 1-3.5 | 1.5-3 | 2-2.5 | 60-95 | 65-90 | 70-85 |
| Second super | 0.2-0.1 | 0.04-0.08 | 0.05-0.06 | 0.5-10 | 1.5-7 | 2.5-5 |
| absorbent layer | | | | | | |
| Second non- | 0.1-0.7 | 0.3-0.5 | 0.4-0.45 | 5-20 | 7-15 | 10-12 |
| woven distribution | | | | | | |
| layer | | | | | | |

### Example I

Example I prepared according to the present invention includes a 0.1 mm thick top sheet, an SAP Matrix with a thickness of 3.4 mm, a pulp layer with a thickness of 8 mm, and a backing layer with a thickness of 0.1 mm. The SAP matrix includes a first non-woven distribution layer with a thickness of 0.4 mm, a first SAP layer with a thickness of 0.05 mm, a cellulosic layer made from cotton with a thickness of 2.5 mm, a second SAP layer with a thickness of 0.05 mm, and a second non-woven distribution layer with a thickness of 0.4 mm. The SAP Matrix includes a first non-woven distribution layer prepared from spunbound, a first SAP layer including superabsorbent polymers wherein the polymers are acrylic acid copolymers, or polymers of acrylic acid, a cellulosic layer prepared from cotton, a second SAP layer including superabsorbent polymers wherein the polymers are acrylic acid copolymers, or polymers of acrylic acid, and a second non-woven distribution layer prepared from spunbound material. The pulp layer includes total chlorine free cellulosic fluff pulp and the backing layer is a spunbound nonwoven material.

The chart below illustrates a comparison of the diapers prepared according to the present invention (Example 1 detailed above) versus selected currently available commercial products. Where available, both girls' and boys' versions of the respective diapers were tested. The chart illustrates the differences in absorption capacity, rewet and retention capacity.

Absorption capacity is a measurement of the amount of liquid, for example urine, that can be absorbed by the diaper. Generally, this measurement is provided in grams (g) of liquid.

Rewet is a measurement of liquid that returns to the surface of a diaper when the diaper is subject to pressure. Rewet is measured in grams (g).

Retention capacity is a measurement of the grams of fluid absorbed by the diaper that cannot be released under pressure. Retention capacity is measured in grams/grams (g/g)

The analysis was conducted by SGS North America, Inc. in accordance with the NWSP Standard Test Methods. SGS is a leading inspection, verification, testing and certification company specialized in diaper performance.

TABLE III

| | Absorption | Rewet | Retention |
|---|---|---|---|
| Diaper Products | Capacity (g) | (g) | Capacity (g/g) |
| Example I | 986 | 0.033 | 467 |
| COTERIE ® | 906 | 0.604 | 448 |
| PAMPERS PURE ® | 622 | 0.038 | 285 |
| RASCAL + FRIENDS ® | 942 | 0.269 | 401 |
| HONEST COMPANY ® | 678 | 0.031 | 371 |
| MILLE MOON ® | 1044 | 0.158 | 383 |
| PAMPERS ® | 614 | 0.044 | 287 |
| HUGGIES LITTLE | 744 | 0.039 | 287 |
| MOVERS ® | | | |

Table III provides a relative comparison of absorption capacity, rewet and retention of the selected commercial products and Example 1 of the present invention. As can be seen in the table below, the relative absorption capacity of Example I is between about 10% and 60% greater than the selected commercial products (with the exception of MILLE MOON®). The relative rewet of the Example I is between 15% and 1800% lower than the selected commercial products (with the exception of HONEST COMPANY®, however this product has a lower absorption capacity and lower retention capacity than Example I according to the study). A lower rewet is better for disposable diapers because it means less liquid returns to the surface when pressure is applied, such as an infant sitting in a wet diaper. Finally, the relative retention capacity of Example I is between about 4% and 64% greater than the selected commercial products.

Embodiments can include a diaper core including a top sheet, a super absorbent polymer matrix, wherein the super absorbent polymer matrix can include a first non-woven distribution layer, a first super absorbent polymer layer, a cellulosic layer, a second super absorbent polymer layer, a second non-woven distribution layer, and a pulp layer.

Embodiment can include the diaper core including a top sheet including apertures configured to assist with the transport of liquid to the super absorbent matrix.

Embodiments can include a diaper wherein the top sheet can be prepared from nonwoven fabrics.

Embodiments can include a diaper core wherein the top sheet can have a thickness between 0.09 mm and 0.1 mm mm.

Embodiments can include a diaper core wherein the first super absorbent polymer layer has a thickness between 0.02 mm and 0.08 mm.

Embodiments can include a diaper core wherein the first and second super absorbent polymer layers can include polymers selected from the group consisting of acrylic acid copolymers, saponified acrylic acid ester-vinyl acetate copolymer, hydrolyzed starch-acrylonitrile graft copolymer, acrylamide copolymer, neutralized starch-acrylic acid graft copolymer, hydrolyzed acrylonitrile copolymer, modified cross-linked polyvinyl alcohol, neutralized self-cross-linking polyacrylic acid, cross-linked polyacrylate salt, carboxylated cellulose, cross-linked polyacrylates including sodium polyacrylate and polyacrylamides; graft copolymers on polysaccharides such as chitin, chitosan, cellulose, starch, natural gums and polypeptide-based copolymers such as saponified starch-graft polyacrylonitrile, hydroxypropyl-cellulose; hydroxypropylmethylcellulose, carboxymethyl-cellulose; carboxymethyl starch; cross-linked copolymers of maleic anhydride; polyvinyl alcohol; polyvinyl ethers; polymers and copolymers of vinyl sulfonic acid; copolymer, neutralized crosslinked isobutylene-maleic anhydride copolymer, and mixtures thereof.

Embodiments can include a diaper core wherein the first and second super absorbent polymer layers can include acrylic acid copolymers, polymers of acrylic acid, and mixtures thereof.

Embodiments can include a diaper core wherein the cellulosic layer can include cotton.

Embodiments can include a diaper core wherein the cellulosic layer can have a thickness between 1 mm and 3.5 mm.

Embodiments can include a diaper core wherein the pulp layer can include a material selected from the group including comminuted wood pulp, tissue, tissue wraps, tissue laminates, cotton, creped cellulose wadding, melt blown polymers, chemically stiffened cellulosic fibers, modified cellulosic fibers, cross-linked cellulosic fibers, absorbent foams, absorbent sponges, super absorbent polymers, superabsorbent fibers, absorbent gelling materials, and mixtures thereof.

Embodiments can include a diaper core wherein the pulp layer can include total chlorine free cellulosic fluff pulp.

Embodiments can include a diaper core wherein the pulp layer can have a thickness between 4 mm and 10 mm.

Embodiments can include a diaper core wherein the first super absorbent polymer layer can be between 0.5 and 10 weight percent of the super absorbent polymer matrix.

Embodiments can include a diaper core wherein the second super absorbent polymer layer can be between 0.5 and 10 weight percent of the super absorbent polymer matrix.

Embodiments can include a diaper core wherein the cellulosic layer can include between 60 and 95 weight percent of the super absorbent polymer matrix.

Embodiments can include a diaper core wherein the first and second non-woven distribution layer can individually be between 5 and 20 weight percent of the super absorbent polymer matrix. Embodiments can include a diaper core including a top sheet, wherein the top sheet has a thickness between 0.09 mm and 1.11 mm and is present between 0.08 and 1.2 weight percent of the diaper core, a super absorbent polymer matrix, wherein the super absorbent polymer matrix has a thickness between 2.9 mm and 3.0 mm and is present between 25 and 35 weight percent of the diaper core, a pulp layer, wherein the pulp layer has a thickness between 6 mm and 8 mm and is present between 65 and 70 weight percent of the diaper core. The embodiment can further include the super absorbent polymer matrix including a first non-woven distribution layer, wherein the first non-woven distribution layer has a thickness between 0.4 mm and 0.45 mm and is present between 10 and 12 weight percent of the super absorbent polymer matrix, a first super absorbent polymer layer, wherein the first super absorbent polymer layer has a thickness between 0.05 mm and 0.06 mm and is present between 2.5 and 5 weight percent of the super absorbent polymer matrix, a cellulosic layer, wherein the cellulosic layer has a thickness between 2 mm and 2.5 mm and is present between 70 and 85 weight percent of the super absorbent polymer matrix, a second super absorbent polymer layer, wherein the second super absorbent polymer layer has a thickness between 0.4 mm and 0.45 mm and is present between 10 and 12 weight percent of the super absorbent polymer matrix, a second non-woven distribution layer, wherein the second non-woven distribution layer has a thickness between 0.05 mm and 0.06 mm and is present between 2.5 and 5 weight percent of the super absorbent polymer matrix.

Embodiments can include a diaper core wherein the pulp layer can include total chlorine free cellulosic fluff pulp.

Embodiments can include a method of preparing diaper cores, wherein the diaper cores can include a top sheet, a super absorbent polymer matrix, wherein the super absorbent polymer matrix can include a first non-woven distribution layer, a first super absorbent polymer layer, a cellulosic layer, a second super absorbent polymer layer, a second non-woven distribution layer, and a pulp layer.

A feature illustrated in one of the figures may be the same as or similar to a feature illustrated in another of the figures. Similarly, a feature described in connection with one of the figures may be the same as or similar to a feature described in connection with another of the figures. The same or similar features may be noted by the same or similar reference characters unless expressly described otherwise. Additionally, the description of a particular figure may refer to a feature not shown in the particular figure. The feature may be illustrated in and/or further described in connection with another figure.

Elements of processes (i.e, methods) described herein may be executed in one or more ways such as by a human, by a processing device, by mechanisms operating automatically or under human control, and so forth. Additionally, although various elements of a process may be depicted in the figures in a particular order, the elements of the process may be performed in one or more different orders without departing from the substance and spirit of the disclosure herein.

The foregoing description sets forth numerous specific details such as examples of specific systems, components, methods and so forth, in order to provide a good understanding of several implementations. It will be apparent to one skilled in the art, however, that at least some implementations may be practiced without these specific details. In other instances, well-known components or methods are not described in detail or are presented in simple block diagram format in order to avoid unnecessarily obscuring the present implementations. Thus, the specific details set forth above are merely exemplary. Particular implementations may vary from these exemplary details and still be contemplated to be within the scope of the present implementations.

Related elements in the examples and/or embodiments described herein may be identical, similar, or dissimilar in different examples. For the sake of brevity and clarity, related elements may not be redundantly explained. Instead, the use of a same, similar, and/or related element names and/or reference characters may cue the reader that an element with a given name and/or associated reference character may be similar to another related element with the same, similar, and/or related element name and/or reference character in an example explained elsewhere herein. Elements specific to a given example may be described regarding that particular example. A person having ordinary skill in the art will understand that a given element need not be the same and/or similar to the specific portrayal of a related element in any given figure or example in order to share features of the related element.

It is to be understood that the foregoing description is intended to be illustrative and not restrictive. Many other implementations will be apparent to those of skill in the art upon reading and understanding the above description. The scope of the present implementations should, therefore, be determined with reference to the appended claims, along with the full scope of equivalents to which such claims are entitled.

The foregoing disclosure encompasses multiple distinct examples with independent utility. While these examples have been disclosed in a particular form, the specific examples disclosed and illustrated above are not to be considered in a limiting sense as numerous variations are possible. The subject matter disclosed herein includes novel and non-obvious combinations and sub-combinations of the various elements, features, functions and/or properties disclosed above both explicitly and inherently. Where the disclosure or subsequently filed claims recite “a” element, “a first” element, or any such equivalent term, the disclosure or claims is to be understood to incorporate one or more such elements, neither requiring nor excluding two or more of such elements.

As used herein “same” means sharing all features and “similar” means sharing a substantial number of features or sharing materially important features even if a substantial number of features are not shared. As used herein “may” should be interpreted in a permissive sense and should not be interpreted in an indefinite sense. Additionally, use of “is” regarding examples, elements, and/or features should be interpreted to be definite only regarding a specific example and should not be interpreted as definite regarding every example. Furthermore, references to “the disclosure” and/or “this disclosure” refer to the entirety of the writings of this document and the entirety of the accompanying illustrations, which extends to all the writings of each subsection of this document, including the Title, Background, Brief description of the Drawings, Detailed Description, Claims, Abstract, and any other document and/or resource incorporated herein by reference.

As used herein regarding a list, “and” forms a group inclusive of all the listed elements. For example, an example described as including A, B, C, and D is an example that includes A, includes B, includes C, and also includes D. As used herein regarding a list, “or” forms a list of elements, any of which may be included. For example, an example described as including A, B, C, or D is an example that includes any of the elements A, B, C, and D. Unless otherwise stated, an example including a list of alternatively-inclusive elements does not preclude other examples that include various combinations of some or all of the alternatively-inclusive elements. An example described using a list of alternatively-inclusive elements includes at least one element of the listed elements. However, an example described using a list of alternatively-inclusive elements does not preclude another example that includes all of the listed elements. And, an example described using a list of alternatively-inclusive elements does not preclude another example that includes a combination of some of the listed elements. As used herein regarding a list, “and/or” forms a list of elements inclusive alone or in any combination. For example, an example described as including A, B, C, and/or D is an example that may include: A alone; A and B; A, B and C; A, B, C, and D; and so forth. The bounds of an “and/or” list are defined by the complete set of combinations and permutations for the list.

Where multiples of a particular element are shown in a FIG., and where it is clear that the element is duplicated throughout the FIG., only one label may be provided for the element, despite multiple instances of the element being present in the FIG. Accordingly, other instances in the FIG. of the element having identical or similar structure and/or function may not have been redundantly labeled. A person having ordinary skill in the art will recognize based on the disclosure herein redundant and/or duplicated elements of the same FIG. Despite this, redundant labeling may be included where helpful in clarifying the structure of the depicted examples.

The Applicant(s) reserves the right to submit claims directed to combinations and sub-combinations of the disclosed examples that are believed to be novel and non-obvious. Examples embodied in other combinations and sub-combinations of features, functions, elements and/or properties may be claimed through amendment of those claims or presentation of new claims in the present application or in a related application. Such amended or new claims, whether they are directed to the same example or a different example and whether they are different, broader, narrower or equal in scope to the original claims, are to be considered within the subject matter of the examples described herein.

1. A diaper core comprising:
a top sheet,
a super absorbent polymer matrix,
wherein the super absorbent polymer matrix comprises:
a first non-woven distribution layer,
a first super absorbent polymer layer,
a cellulosic layer,
a second super absorbent polymer layer,
a second non-woven distribution layer, and
a pulp layer.

2. The diaper core according to claim 1,
wherein:
the top sheet comprises apertures configured to assist with the transport of liquid to the super absorbent matrix.

3. The diaper core according to claim 2,
wherein the top sheet is prepared from nonwoven fabrics.

4. The diaper core according to claim 3,
wherein the top sheet has a thickness between 0.09 mm and 0.1 mm.

5. The diaper core according to claim 1,
wherein the first super absorbent polymer layer has a thickness between 0.02 mm and 0.08 mm.

6. The diaper core according to claim 1,
wherein the first and second super absorbent polymer layers comprise polymers selected from the group consisting of:
acrylic acid copolymers, saponified acrylic acid ester-vinyl acetate copolymer, hydrolyzed starch-acrylonitrile graft copolymer, acrylamide copolymer, neutralized starch-acrylic acid graft copolymer, hydrolyzed acrylonitrile copolymer, modified cross-linked polyvinyl alcohol, neutralized self-cross-linking polyacrylic acid, cross-linked polyacrylate salt, carboxylated cellulose, cross-linked polyacrylates including sodium polyacrylate and polyacrylamides; graft copolymers on polysaccharides such as chitin, chitosan, cellulose, starch, natural gums and polypeptide-based copolymers such as saponified starch-graft polyacrylonitrile, hydroxypropyl-cellulose; hydroxypropylmethylcellulose, carboxymethyl-cellulose; carboxymethyl starch; cross-linked copolymers of maleic anhydride; polyvinyl alcohol; polyvinyl ethers; polymers and copolymers of vinyl sulfonic acid; copolymer, neutralized crosslinked isobutylene-maleic anhydride copolymer, and mixtures thereof.

7. The diaper core according to claim 6,
wherein the first and second super absorbent polymer layers comprise acrylic acid copolymers, polymers of acrylic acid, and mixtures thereof.

8. The diaper core according to claim 1,
wherein the cellulosic layer comprises cotton.

9. The diaper core according to claim 8,
wherein the cellulosic layer has a thickness between 1 mm and 3.5 mm.

10. The diaper core according to claim 9,
wherein the pulp layer comprises a material selected from the group including:
comminuted wood pulp, tissue, tissue wraps, tissue laminates, cotton, creped cellulose wadding, melt blown polymers, chemically stiffened cellulosic fibers, modified cellulosic fibers, cross-linked cellulosic fibers, absorbent foams, absorbent sponges, super absorbent polymers, superabsorbent fibers, absorbent gelling materials, and mixtures thereof.

11. The diaper core according to claim 10,
wherein the pulp layer comprises total chlorine free cellulosic fluff pulp.

12. The diaper core according to claim 9,
wherein the pulp layer has a thickness between 4 mm and 10 mm.

13. The diaper core according to claim 1,
wherein the first super absorbent polymer layer comprises between 0.5 and 10 weight percent of the super absorbent polymer matrix.

14. The diaper core according to claim 1,
wherein the second super absorbent polymer layer comprises between 0.5 and 10 weight percent of the super absorbent polymer matrix.

15. The diaper core according to claim 1,
wherein the cellulosic layer comprises between 60 and 95 weight percent of the super absorbent polymer matrix.

16. The diaper core according to claim 1,
wherein the first non-woven distribution layer comprises between 5 and 20 weight percent of the super absorbent polymer matrix.

17. The diaper core according to claim 1,
wherein the second non-woven distribution layer comprises between 5 and 20 weight percent of the super absorbent polymer matrix.

18. A diaper core comprising:
a top sheet,
wherein the top sheet has a thickness between 0.09 mm and 1.11 mm and is present between 0.08 and 1.2 weight percent of the diaper core,
a super absorbent polymer matrix,
wherein the super absorbent polymer matrix has a thickness between 2.9 mm and 3.0 mm and is present between 25 and 35 weight percent of the diaper core,
wherein the super absorbent polymer matrix comprises:
a first non-woven distribution layer,
wherein the first non-woven distribution layer has a thickness between 0.4 mm and 0.45 mm and is present between 10 and 12 weight percent of the super absorbent polymer matrix,
a first super absorbent polymer layer,
wherein the first super absorbent polymer layer has a thickness between 0.05 mm and 0.06 mm and is present between 2.5 and 5 weight percent of the super absorbent polymer matrix,
a cellulosic layer,
wherein the cellulosic layer has a thickness between 2 mm and 2.5 mm and is present between 70 and 85 weight percent of the super absorbent polymer matrix,
a second super absorbent polymer layer,
wherein the second super absorbent polymer layer has a thickness between 0.4 mm and 0.45 mm and is present between 10 and 12 weight percent of the super absorbent polymer matrix,
a second non-woven distribution layer,
wherein the second non-woven distribution layer has a thickness between 0.05 mm and 0.06 mm and is present between 2.5 and 5 weight percent of the super absorbent polymer matrix, and
a pulp layer,
wherein the pulp layer has a thickness between 6 mm and 8 mm and is present between 65 and 70 weight percent of the diaper core.

19. The diaper core according to claim 18,
wherein the pulp layer includes total chlorine free cellulosic fluff pulp.

20. A method of preparing diaper cores,
wherein the diaper cores comprise:
a top sheet,
a super absorbent polymer matrix,
wherein the super absorbent polymer matrix comprises:
a first non-woven distribution layer,
a first super absorbent polymer layer,
a cellulosic layer,
a second super absorbent polymer layer,
a second non-woven distribution layer, and
a pulp layer.
